(19) United States
(12) Patent Application Publication
(10) Pub. No.: US 2006/0105466 A1
(43) Pub. Date: May 18, 2006

(54) Sensor for determining concentration of ozone

(75) Inventors: Michael A. Centanni, Parma, OH (US); Peter A. Burke, Concord, OH (US)

Correspondence Address: KUSNER & JAFFE;HIGHLAND PLACE SUITE 310, 6151 WILSON MILLS ROAD, HIGHLAND HEIGHTS, OH, 44143, US

(73) Assignee: STERIS Inc.

(21) Appl. No.: 10/987,072

(22) Filed: Nov. 12, 2004

(51) Int. Cl. A61L 2/20; G01N 33/00

(52) U.S. Cl. 436/135; 422/62; 422/82.02

(57) ABSTRACT

The present invention discloses a sensor for detecting ozone, the sensor comprises an element exhibiting piezoelectric properties having a coating that is removed from the quartz crystal upon exposure to ozone.

### FIELD OF THE INVENTION

The present invention relates generally to decontamination systems, and more particularly to a sensor for determining the concentration of ozone in a defined region.

### BACKGROUND OF THE INVENTION

Sterilization and decontamination methods are used in a broad range of applications, and have used an equally broad range of sterilization agents. As used herein the term “sterilization” refers to the inactivation of all bio-contamination, especially on inanimate objects. The term “disinfectant” refers to the inactivation of organisms considered pathogenic.

In general, sterilization/decontamination systems rely on maintaining certain process parameters in order to achieve a target sterility or decontamination assurance level. For example, maintaining a set concentration of the sterilant/decontaminant, e.g., ozone, within a region where such sterilization/decontamination is to be effected works to achieve a target sterility or decontamination assurance level. In this respect, it is desirable that measurements of the concentration of sterilants such as ozone be made in real time as a sterilization or decontamination process proceeds.

The present invention provides a sensor for detecting the concentration of ozone in a defined region.

### SUMMARY OF THE INVENTION

In accordance with one aspect of the present invention, there is provided a sensor for detecting ozone, comprising a substrate exhibiting piezoelectric properties having first and second major surfaces. A first electrode is connected to the first major surface and a second electrode connected to the second major surface. A layer of a material is provided on at least one of the first and second major surfaces. The material is operable to decrease the mass of the sensor when exposed to ozone.

In accordance with another aspect of the present invention, there is provided a sensor for detecting ozone, comprising an element exhibiting piezoelectric properties having a material that is removed from the quartz crystal upon exposure to ozone.

In accordance with a preferred embodiment of the present invention, there is provided a sensor for detecting ozone, comprising an element exhibiting piezoelectric properties that supports a carbon-containing material.

In accordance with another aspect of the present invention, there is provided a method of determining the presence of a ozone in a region of a decontamination system having a chamber defining the region and a circulation system for supplying the ozone to the region, comprising the steps of: providing in said region an element having piezoelectric properties with a coating that reacts with ozone; determining a baseline frequency of oscillation for said element in the absence of ozone; determining a measured frequency of oscillation for said element when exposed to ozone in said region, said measured frequency being greater than said baseline frequency; and, determining the concentration of ozone in said region based upon said measured frequency.

In accordance with yet another aspect of the present invention, there is provided a system for the deactivation of bio-contamination or chemical contamination, comprising: a system for moving ozone through a space; a piezoelectric device that supports a coating including a material that reacts with ozone, said piezoelectric device having a measured frequency that increases over a baseline frequency in response to the presence of said ozone; and a controller having data stored therein relating to said piezoelectric device, said data relating an increased frequency of said piezoelectric device to a concentration of said ozone.

In accordance with still another aspect of the present invention, there is provided a method of determining a concentration of a sterilant in a region of a decontamination system having a chamber defining the region and a circulation system for supplying the sterilant to the region, comprising the steps of: providing in said region a piezoelectric device having a coating including a material that reacts stoichiometrically with the sterilant; determining a baseline frequency of oscillation for said piezoelectric device in the absence of the sterilant; exposing said piezoelectric device to the sterilant; and determining a slope of a frequency versus time curve generated when said piezoelectric device is exposed to the sterilant and comparing said slope with stored slopes of frequency versus time curves obtained when said piezoelectric device is exposed to different concentrations of the sterilant, and thereby determining the concentration of the sterilant in the region.

In accordance with still another aspect of the present invention, there is provided a system for deactivation of bio-contamination or chemical contamination, comprising: a system for moving a sterilant through a space; a piezoelectric device that supports a coating including a material that reacts stoichiometrically with the sterilant, said piezoelectric device having a measured frequency that increases over a baseline frequency in response to the presence of the sterilant; and a controller having data stored therein, said data comprising slopes of frequency versus time curves obtained when said piezoelectric device is exposed to different concentrations of the sterilant.

An advantage of the present invention is a sensor for determining the concentration of ozone in a region of space.

Another advantage of the present invention is a sensor as described above that can determine the concentration of ozone, during the course of a decontamination cycle.

These and other advantages will become apparent from the following description of a preferred embodiment taken together with the accompanying drawings and the appended claims.

### BRIEF DESCRIPTION OF THE DRAWINGS

The invention may take physical form in certain parts and arrangement of parts, a preferred embodiment of which will be described in detail in the specification and illustrated in the accompanying drawings which form a part hereof, and wherein:

FIG. 1 is a schematic view of a decontamination system;

FIG. 2 is a top, plan view of a sensor for determining the concentration of ozone as used in a decontamination system;

FIG. 3 is a side, elevation view of the sensor shown in FIG. 2;

FIG. 4 is an exploded view of the sensor shown in FIG. 2;

FIG. 5 is a graph that illustrates the frequency versus time of a quartz crystal coated with a fugitive material that is exposed to ozone; and,

FIG. 6 displays a family of frequency versus time curves, each curve illustrating the response of a quartz crystal coated with a fugitive material wherein each curve is exposed to a different concentration of ozone.

### DETAILED DESCRIPTION OF PREFERRED EMBODIMENT

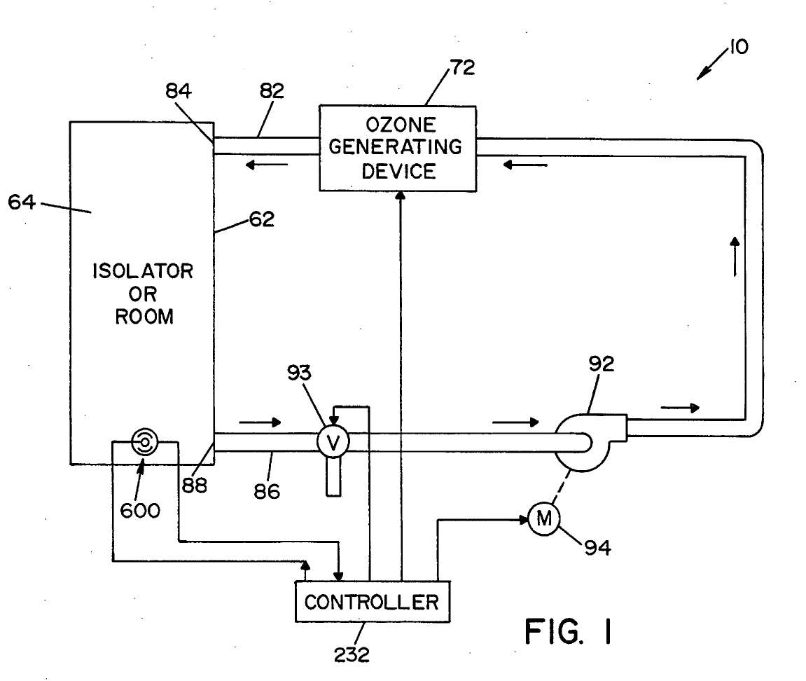

Referring now to the drawings wherein the showings are for the purpose of illustrating a preferred embodiment of the invention only, and not for the purpose of limiting same, FIG. 1 shows a decontamination system 10 having a sensor 600 for determining the concentration of ozone as used within system 10. In the embodiment shown, system 10 is a closed-loop decontamination system for decontaminating objects with ozone. Accordingly, sensor 600 shall be described with respect to determining the concentration of ozone within such a closed-loop decontamination system.

In the embodiment shown, system 10 includes an isolator or room 62 that defines an inner sterilization/decontamination chamber or region 64. It is contemplated that articles to be sterilized or decontaminated may be disposed within inner sterilization/decontamination chamber or region 64 of isolator or room 62. An ozone generating device 72 is connected to inner sterilization/decontamination chamber or region 64 of room or isolator 62 by means of a supply conduit 82. Ozone generating device 72 may consist of a source of ultraviolet light exposed to an oxygen-containing gas or a charged capacitor through which an oxygen containing gas passes. Optionally, ozone may be introduced into supply conduit 82 from an external source or generator. Supply conduit 82 defines an inlet 84 to inner sterilization/decontamination chamber or region 64 of room or isolator 62.

Isolator or room 62 is part of a closed loop system that includes a return conduit 86 that connects an outlet port 88 to isolator or room 62 (and inner sterilization/decontamination chamber or region 64) to a blower 92. Blower 92, driven by a motor 94, is disposed within return conduit 86 between isolator or room 62 and ozone generating device 72. Blower 92 is operable to circulate ozone and a carrier gas, preferably air or an oxygen-containing gas, through the closed loop system. Optionally, a filter may be disposed in supply conduit 82 between blower 92 and ozone generating device 72. A valve 93 is disposed in return conduit 86. Valve 93 opens to the atmosphere, thus providing a means to vent system 10 of ozone after a decontamination cycle is run.

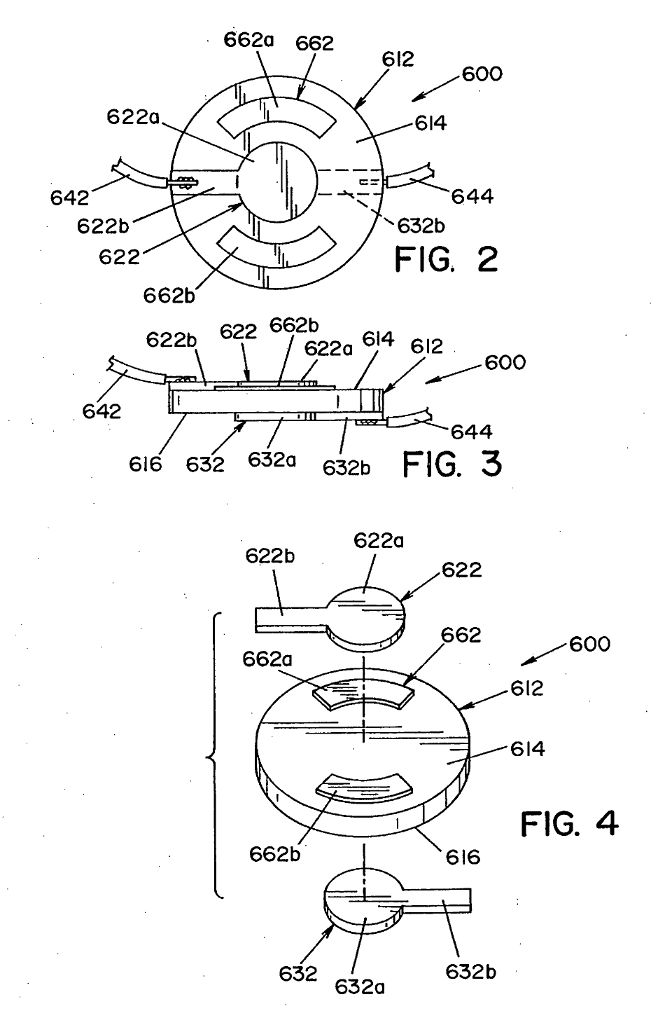

A sensor 600 is disposed within inner sterilization/decontamination chamber or region 64 to sense and/or monitor the concentration of ozone therein. Sensor 600 is best seen in FIGS. 2-4. Broadly stated, sensor 600 is comprised of an element 612 having a layer or coating 662 of a material that interacts with, or is reactive with, the ozone used in system 10, such that mechanical motion or movement of element 612 is converted into an electrical signal.

Element 612 may be a moving or suspended component, but in a preferred embodiment, element 612 is a piezoelectric device, and more preferably, is a quartz crystal. Typical quartz crystals that may be used have resonant frequencies of oscillation of 5 Megahertz or 10 Megahertz (MHz). Other piezoelectric materials, such as by way of example and not limitation, Rochelle salt, barium titanate, tourmaline, polyvinylidene fluoride or crystals that lack a center of symmetry are also contemplated. In the embodiment shown, element 612 is a flat, circular quartz disk having a first planar, major surface 614 and a second planar, major surface 616. An electrode 622 is disposed on the first planar, major surface 614 and an electrode 632 is disposed optionally on the second planar, major surface 616.

Electrode 622 includes a main body portion 622a that is centrally disposed on first major surface 614 and a leg portion 622b that extends in a first direction to the edge of element 612. Similarly, electrode 632 includes a main body portion 632a that is centrally disposed on second major, planar surface 616, and a leg portion 632b that extends in a direction opposite to the first direction of leg portion 622b, wherein leg portion 632b extends to the edge of element 612. Main body portions 622a, 632a of electrodes 622, 632 are disposed respectively on first and second major surfaces 614, 616 to be aligned with each other on opposite sides of element 612. Leg portions 622b, 632b extend in opposite directions from central body portions 622a, 632a, as best seen in the drawings. Electrodes 622, 632 are deposited onto first and second planar surfaces 614, 616. Electrodes 622, 632 may be formed of any electrically conductive material, but are preferably formed of copper, silver or gold. Electrical leads 642, 644 are attached to leg portions 622b, 632b of electrodes 622, 632. Leads 642, 644 are soldered, braised or welded to electrodes 622, 632 to be in electrical contact therewith.

At least one of the two major surfaces 614, 616 of element 612 is coated with a layer 662 of a material that interacts with, or is reactive with the ozone used within system 10. In the embodiment shown, layer 662 is on major surface 614. In the embodiment shown, layer 662 is defined by two arcuate or crescent-shaped layer areas 662a, 662b of material applied to first major surface 614 of element 612. Arcuate layer areas 662a, 662b are disposed on first major surface 614 such that electrode 622 is disposed therebetween. The material forming layer areas 662a, 662b are preferably fixedly attached to surface 614 of element 612. As will be appreciated from a further description of the present invention, the mass of the material on element 612 is dependent upon the desired performance characteristics of sensor 600. As indicated above, the material forming layer areas 662a, 662b are preferably one that interacts or reacts with the ozone used within system 10. In one embodiment, a source of heat is applied to sensor 600 to catalyze the reaction between layer areas 662a, 662b and ozone. Examples of such heat sources include, but are not limited to, resistance heat, i.e., joule heat produced by a heated wire, and infrared heat.

In a preferred embodiment of the present invention, the sterilant to be detected is ozone (O₃), and the material that forms layer areas 662a, 662b on first major surface 614 of sensor 600 is a carbon-containing material. For example, the carbon-containing material could include a thin coating of a polymer having carbon particulate adhered thereto or the carbon particulate could be dispersed throughout the polymer film, the polymer film acting as a matrix for the carbon particulate. In one embodiment, the carbon is activated carbon. In one embodiment, the polymer matrix is formed of an amorphous polymer thus affording easy ingress of the ozone gas to the carbon particulate dispersed therein—as the amorphous region of the polymer matrix is “spongy” in nature, the ozone gas easily diffuses into the polymer matrix.

Layer areas 662a, 662b are preferably formed by a thin film deposition process. For example, carbon particulate could be dispersed in a solvated polymer solution and then spin cast on the surface of the quartz crystal. In another deposition technique, a thin polymer coating could be placed on the quartz crystal and while wet, dusted with carbon particulate. Upon drying, the polymer adheres the carbon particulate to the surface of the quartz crystal.

Sensor 600 is disposed within inner sterilization/decontamination chamber or region 64, and is connected to a system controller 232, that is schematically illustrated in FIG. 1, to provide electrical signals thereto. Controller 232 is a system microprocessor or microcontroller programmed to control the operation of system 10. As illustrated in FIG. 1, controller 232 is also connected to motor 94. Controller 232 includes an oscillating circuit (not shown) that is connected to sensor 600 to convert movement of sensor 600 into electrical signals, as is conventionally known. In another embodiment, the oscillating circuit may be located at the site of the quartz crystal. In yet another embodiment, controller 232 may control and receive data from the quartz crystal by radio waves, with appropriate receivers and transmitters stationed at the site of the quartz crystal and controller 232. Controller 232 also includes stored data indicative of the electrical responses of sensor 600 to predetermined concentrations of the ozone to be sensed. In the embodiment heretofore described, where element 612 is a quartz crystal and layer areas 662a, 662b are carbon-containing materials, the data relating to sensor 600 that is stored within controller 232 is empirical data accumulated under controlled, laboratory conditions.

In accordance with the present invention, the empirical data relating to sensor 600 that is stored in controller 232 may be acquired as follows. The natural frequency of a quartz crystal (without a coating thereon) is measured. The carbon-containing material is applied to the quartz crystal and a baseline frequency or mass of the coating is determined using the Sauerbre equation. The quartz crystal is then exposed to various, controlled concentrations of ozone. The frequency versus time curves obtained thereby are stored in controller 232.

In one embodiment, the change in frequency or weight is divided by the mass of the coating applied to the quartz crystal so that regardless of the mass of coatings applied to other crystals, the change in frequency will be normalized to a unit mass of the coating. Data taken with other quartz crystals that may have coatings of different amounts of mass than the laboratory crystal can still be compared to the stored data obtained from the laboratory crystal as both sets of data will be normalized to a change in frequency or weight per unit mass of the coating.

In another embodiment, a quartz crystal is coated with a carbon-containing material and is then exposed to known concentrations of ozone so as to develop a set of curves, or data, of frequency as a function of time. All of these curves will show an increase in frequency of the quartz crystal as the carbon-containing quartz crystal is exposed to ozone. The coated quartz crystal is then installed in a system 10. The associated set of data, or curve, is programmed or stored in controller 232 of system 10. Thus, the data stored in system 10 matches the crystal sensor within system 10, thereby providing a standardized sensor system. In this manner, each system 10 has a coated quartz crystal sensor with an associated standardized data set therein, as the stored data set was produced by exposing that specific quartz crystal to known concentrations of ozone.

It will be appreciated that ozone gas reacts with the carbon of the carbon-containing material to form carbon dioxide and carbon monoxide thus removing the carbon of the carbon-containing material from the surface of the quartz crystal. The removal of the carbon of the carbon-containing material from the surface of the quartz crystal results in an increase in the frequency of oscillation of the quartz crystal. Parenthetically, after all of the carbon of the carbon-containing material is consumed, the quartz crystal will need to be replaced.

The present invention shall now be further described with reference to the operation of system 10. Sensor 600 operates based upon the concept that the frequency of a piezoelectric device will change in relation to a change in mass of a layer on the device, as a result of exposure to ozone.

Specifically, the frequency of a piezoelectric device is related to the mass change, as determined by the Sauerbre equation:
Δf=−(C_{f})(Δm)
Δf=−(fₒ²/Nρ)Δm
where:
Δf is the frequency change;
Δm is the mass change per unit area on the surface of the piezoelectric device;
C_{f} is a sensitivity constant;
fₒ is the operating frequency of the piezoelectric device prior to the mass change;
N is the frequency constant for the piezoelectric device; and,
ρ is the density of the piezoelectric device.

Isolator or room 62, supply conduit 82 and return conduit 86 define a closed loop conduit circuit. When a sterilization/decontamination cycle is first initiated, controller 232 initiates ozone generating device 72 and causes blower motor 94 to drive blower 92, thereby initiating the generation of ozone and causing a carrier gas to circulate through the closed loop circuit. In the embodiment shown, the carrier gas is air. In another embodiment, the carrier gas could be oxygen or an oxygen-containing stream of gas. Ozone generating device 72 may produce ozone by exposing oxygen in the carrier gas to ultraviolet light or to a charged capacitor. Ozone carried by the carrier gas is introduced into isolator or room 62 through supply conduit 82 and inlet 84. Controller 232 controls blower motor 94 such that the residence time of the ozone within isolator or room 62 is sufficient to decontaminate isolator or room 62 and the items located therein. Controller 232 also controls ozone generating device 72 so that appropriate levels of ozone are generated thus assuring proper decontamination of inner sterilization/decontamination chamber or region 64 and items located therein. In turn, sensor 600 provides an electrical signal to controller 232 indicative of the concentration of ozone within inner sterilization/decontamination chamber or region 64 of isolator or room 62 to assure that the concentration level of ozone, for its residence time, is sufficient to decontaminate inner sterilization/decontamination chamber or region 64 of isolator or room 62 and the items located therein.

After the decontamination phase, an aeration phase is initiated thus bringing the residual ozone level down to an allowable threshold. In this respect, as will be appreciated, ozone generating device 72 is turned off and blower 92 continues to circulate the air through the system. During the aeration phase, valve 93 in return conduit 86 is opened allowing ozone to be vented to the atmosphere.

As illustrated in FIG. 1, sensor 600 is exposed to the atmosphere within inner sterilization/decontamination chamber or region 64 of isolator or room 62. After the aeration phase of system 10, a new operating frequency fₒ′ of sensor 600 is determined by controller 232. Since carbon from the carbon-containing material is removed from the surface of the coated quartz crystal, a new operating frequency fₒ′ of sensor 600 must be determined prior to running a new decontamination cycle. Operating frequency fₒ′ is essentially a new baseline frequency of sensor 600 prior to any mass change that would result from the exposure of sensor 600 to ozone. During a decontamination cycle, sensor 600 is exposed to ozone entering inner sterilization/decontamination chamber or region 64 of isolator or room 62. The ozone will react stoichiometrically with the carbon in accordance with the two chemical equations set forth below:
3C₍ₛ₎+2O_{3(g)}→3CO_{2(g)}; and,
3C₍ₛ₎+O_{3(g)}→3CO_{(g)}.

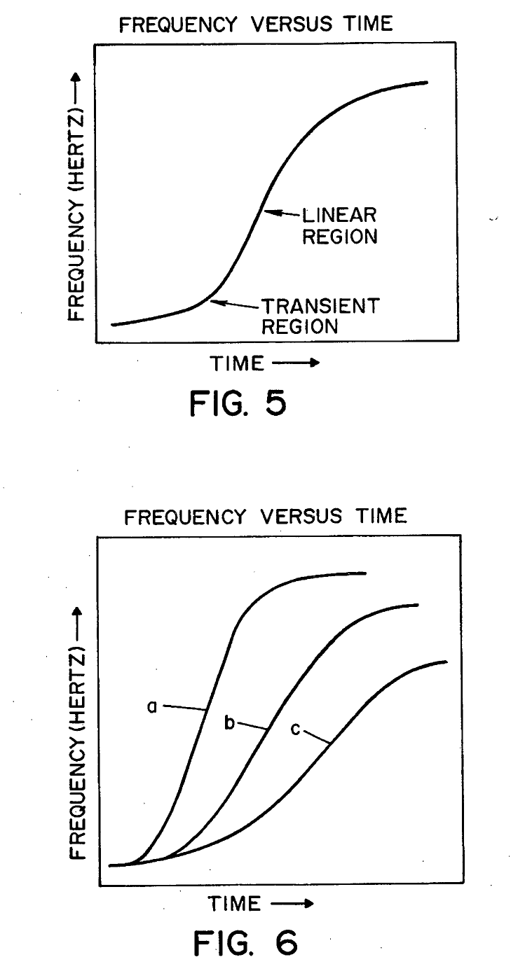

At a fixed concentration of ozone, the frequency of the quartz crystal, as a function of time, will continue to increase with reasonably constant slope (see FIG. 5) as the ozone present reacts with the carbon in or on the coating forming carbon dioxide and carbon monoxide. Thus, even at constant concentration of ozone, carbon is continually removed from the surface of the quartz crystal resulting in a constant increase in the frequency of oscillation of the coated quartz crystal. Other such fugitive coatings are also contemplated wherein the coating reacts with ozone, is continually removed from the surface of the quartz crystal and wherein the frequency of the quartz crystal after reacting with ozone is greater than a baseline frequency. Frequency/(mass of coating) versus time graphs (or, using the Sauerbre equation, weight/(mass of coating) versus time graphs) for various concentrations of ozone are produced and stored in a data storage device within controller 232. Alternatively, the data could be stored not as a graph but rather in look up tables. As will be appreciated, if a coating of uniform thickness is applied to a crystal, the change in frequency or weight could be normalized on a per unit surface area basis.

Changes in the concentration of ozone are thus realized by changes in the slope of a graph of the frequency versus time curve. Graphs of the frequency as a function of time for a variety of concentrations of ozone exposed to the carbon coated quartz crystal will thus exhibit different slopes for the linear regions and transient regions of the curves. For example, in FIG. 6, curve (a) illustrates a larger concentration of ozone in inner sterilization/decontamination chamber or region 64 of isolator or room 62 than curve (b). Curve (b) illustrates a larger concentration of ozone in inner sterilization/decontamination chamber or region 64 of isolator or room 62 than the concentration of ozone as represented by curve (c).

During an actual decontamination run, the slope of the approximately linear region of the frequency as a function of time curve is compared to the stored curves. A match between the slope of a stored curve and the actual data results in a determination of the concentration of ozone. In addition, it is believed that the transient region, i.e., the region in time prior to the approximately linear region of the frequency versus time curve as illustrated in FIG. 5, may also be used to determine the concentration of ozone when compared to similar regions of the stored curves. Through the use of appropriate software, the stored curves (data) can also be interpolated and/or extrapolated to determine the slopes of the linear regions or other portions of the frequency versus time curves not actually taken under the controlled, laboratory conditions.

The slope and changes in slope of the concentration versus time curve provides the data necessary to determine the concentration of ozone in inner sterilization/decontamination chamber or region 64 of isolator or room 62. Regardless of the coating, the determination of the concentration of ozone within a region of space may be assessed quickly by evaluating the change in the frequency versus time curve of the coated quartz crystal under non-equilibrium conditions. In other words, one does not have to wait until the frequency of the coated quartz crystal comes to equilibrium with the ozone gas before a determination of the concentration of ozone gas can be established. In an actual decontamination process, as soon as the slope of the frequency versus time curve begins to change from a first slope to a second slope, controller 232 searches for a graph that correlates to the measured changes. As soon as controller 232 finds the graph with the measured changes, the concentration of ozone has been determined. Thus, determination of the concentration of ozone under non-equilibrium conditions reduces the time required to make such a determination. Stated another way, it is a change in the first derivative of the frequency versus time curve that signals a change in the concentration of ozone within inner sterilization/decontamination chamber or region 64 of isolator or room 62. Hence, one could store the first derivatives of the transient region or linear region (see FIG. 5) of the frequency versus time curves and correlate the different derivatives to set concentrations of ozone.

The reaction between the carbon-containing material of layer areas 662a, 662b and the ozone (O₃) produces a change (reduction) in the mass of layer areas 662a, 662b. The reduction in mass of sensor 600 results in a change in the operating frequency fₒ thereof. Controller 232 monitors the frequency to determine “measured frequencies” fₘ during a decontamination cycle and during the aeration cycle. In the absence of ozone, the frequency of the coated quartz crystal would remain constant. In each decontamination run, the measured frequencies of each new run fₘ, fₘ′, fₘ″, fₘ′″ . . . are compared to the respective baseline operating frequency fₒ, f₀′, fₒ″, fₒ′″ . . . of each new run to determine a change in frequency as a function of time. For each run, the measured frequency is always greater than the baseline frequency after exposure to ozone as the carbon of carbon-containing coating is a fugitive material, i.e., the carbon of the carbon-containing coating is removed from the quartz crystal as it reacts with ozone. Controller 232 then determines the concentration of ozone within inner sterilization/decontamination chamber or region 64 of isolator or room 62 as indicated hereinabove. Controller 232 is thus able to determine the concentration of ozone (O₃) within inner sterilization/decontamination chamber or region 64 of isolator or room 62 over a short period of time, i.e., the time necessary for controller 232 to determine the slope of the frequency versus time curve and compare it to the slopes of the stored curves. Thus, the concentration of ozone (O₃) within inner sterilization/decontamination chamber or region 64 of isolator or room 62 can be sensed and continuously monitored, based upon a change in frequency of sensor 600.

It will be appreciated that other coatings can be used to detect ozone. For example, polymeric coatings that would be removed from a quartz crystal by reaction with ozone may be used. In one embodiment, it is believed that polymeric coatings with unsaturation can be used as the double bonds are prone to attack by ozone. In one embodiment, the unsaturated polymeric coating is of low molecular weight thus facilitating easier removal of fragments of the polymer chain that have been released from the polymeric chain after exposure to ozone gas.

It will be further appreciated that the sensor 600 may be used in a gaseous environment or a liquid environment to detect, monitor and control the concentration of ozone gas.

The foregoing description is a specific embodiment of the present invention. It should be appreciated that this embodiment is described for purposes of illustration only, and that numerous alterations and modifications may be practiced by those skilled in the art without departing from the spirit and scope of the invention. It is intended that all such modifications and alterations be included insofar as they come within the scope of the invention as claimed or the equivalents thereof.

Other modifications and alterations will occur to others upon their reading and understanding of the specification. It is intended that all such modifications and alterations be included insofar as they come within the scope of the invention as claimed or the equivalents thereof.

Having described the invention, the following is claimed:

1. A method of determining a concentration of ozone in a region of a decontamination system having a chamber defining the region and a circulation system for supplying the ozone to the region, comprising the steps of:
providing in said region an element having piezoelectric properties with a coating that reacts with ozone;
determining a baseline frequency of oscillation for said element in the absence of ozone;
determining a measured frequency of oscillation for said element when exposed to ozone in said region, said measured frequency being greater than said baseline frequency; and,
determining the concentration of ozone in said region based upon said measured frequency.

2. The method of claim 1, wherein said coating includes carbon.

3. The method of claim 1, wherein said coating includes a polymer with unsaturation.

4. The method of claim 1, wherein said element is a crystal that lacks a center of symmetry.

5. The method of claim 4, wherein said crystal is a quartz crystal.

6. The method of claim 5, wherein said quartz crystal has a resonant frequency of 5 MHz or 10 MHz.

7. The method of claim 1, further comprising the steps of determining a slope of a frequency versus time curve and comparing said slope with stored slopes of frequency versus time curves for different concentrations of ozone and thereby determining the concentration of ozone.

8. The method of claim 1, wherein said element is one of a quartz crystal, Rochelle salt, barium titanate, tourmaline and polyvinylidene fluoride.

9. A system for the deactivation of bio-contamination or chemical contamination, comprising:
a system for moving ozone through a space;
a piezoelectric device that supports a coating including a material that reacts with ozone, said piezoelectric device having a measured frequency that increases over a baseline frequency in response to the presence of said ozone; and
a controller having data stored therein relating to said piezoelectric device, said data relating an increased frequency of said piezoelectric device to a concentration of said ozone.

10. The system of claim 9, wherein said coating includes carbon.

11. The system of claim 9, wherein said coating includes a polymer with unsaturation.

12. The system of claim 9, wherein said piezoelectric device is a crystal that lacks a center of symmetry.

13. The system of claim 12, wherein said crystal is a quartz crystal.

14. The system of claim 13, wherein said quartz crystal has a resonant frequency of 5 MHz or 10 MHz.

15. The system of claim 9, wherein said piezoelectric device is one of a quartz crystal, Rochelle salt, barium titanate, tourmaline and polyvinylidene fluoride.

16. A sensor for detecting ozone, comprising an element exhibiting piezoelectric properties having a material that is removed from the quartz crystal upon exposure to ozone.

17. The sensor of claim 16, wherein said coating includes carbon.

18. The sensor of claim 16, wherein said coating includes a polymer with unsaturation.

19. The sensor of claim 16, wherein said element is a crystal that lacks a center of symmetry.

20. The sensor of claim 19, wherein said crystal is a quartz crystal.

21. The sensor of claim 20, wherein said quartz crystal has a resonant frequency of 5 MHz or 10 MHz.

22. The sensor of claim 16, wherein said element is one of a quartz crystal, Rochelle salt, barium titanate, tourmaline and polyvinylidene fluoride.

23. The sensor of claim 16, further comprising a source of heat that heats the sensor.

24. A method of determining a concentration of a sterilant in a region of a decontamination system having a chamber defining the region and a circulation system for supplying the sterilant to the region, comprising the steps of:
providing in said region a piezoelectric device having a coating including a material that reacts stoichiometrically with the sterilant;
determining a baseline frequency of oscillation for said piezoelectric device in the absence of the sterilant;
exposing said piezoelectric device to the sterilant; and
determining a slope of a frequency versus time curve generated when said piezoelectric device is exposed to the sterilant and comparing said slope with stored slopes of frequency versus time curves obtained when said piezoelectric device is exposed to different concentrations of the sterilant, and thereby determining the concentration of the sterilant in the region.

25. The method of claim 24, wherein said piezoelectric device is a quartz crystal.

26. The method of claim 25, wherein said quartz crystal has a resonant frequency of 5 MHz or 10 MHz.

27. The method of claim 24, wherein said piezoelectric device is one of a quartz crystal, Rochelle salt, barium titanate, tourmaline and polyvinylidene fluoride.

28. A system for deactivation of bio-contamination or chemical contamination, comprising:
a system for moving a sterilant through a space;
a piezoelectric device that supports a coating including a material that reacts stoichiometrically with the sterilant, said piezoelectric device having a measured frequency that increases over a baseline frequency in response to the presence of the sterilant; and
a controller having data stored therein, said data comprising slopes of frequency versus time curves obtained when said piezoelectric device is exposed to different concentrations of the sterilant.

29. The system of claim 28, wherein said piezoelectric device is a quartz crystal.

30. The system of claim 29, wherein said quartz crystal has a resonant frequency of 5 MHz or 10 MHz.

31. The system of claim 28, wherein said piezoelectric device is one of a quartz crystal, Rochelle salt, barium titanate, tourmaline and polyvinylidene fluoride.
